(19) United States
(12) Patent Application Publication
(10) Pub. No.: US 2021/0291181 A1
(43) Pub. Date: Sep. 23, 2021

(54) SEAL COMPONENT FOR A RAPID DIAGNOSTIC TEST

(71) Applicant: Detect, Inc., Guilford, CT (US)

(72) Inventors: Jonathan M. Rothberg, Guilford, CT (US); Spencer Glantz, West Hartford, CT (US); Benjamin Rosenbluth, Hamden, CT (US)

(73) Assignee: Detect, Inc., Guilford, CT (US)

(21) Appl. No.: 17/203,562

(22) Filed: Mar. 16, 2021

Related U.S. Application Data
(60) Provisional application No. 63/081,201, filed on Sep. 21, 2020.
(60) Provisional application No. 63/068,303, filed on Aug. 20, 2020.
(60) Provisional application No. 63/065,131, filed on Aug. 13, 2020.
(60) Provisional application No. 63/061,072, filed on Aug. 4, 2020.
(60) Provisional application No. 63/059,928, filed on Jul. 31, 2020.
(60) Provisional application No. 63/053,534, filed on Jul. 17, 2020.
(60) Provisional application No. 63/036,887, filed on Jun. 9, 2020.
(60) Provisional application No. 63/034,901, filed on Jun. 4, 2020.
(60) Provisional application No. 63/027,859, filed on May 20, 2020.
(60) Provisional application No. 63/027,874, filed on May 20, 2020.
(60) Provisional application No. 63/027,890, filed on May 20, 2020.
(60) Provisional application No. 63/027,864, filed on May 20, 2020.
(60) Provisional application No. 63/027,878, filed on May 20, 2020.
(60) Provisional application No. 63/027,886, filed on May 20, 2020.
(60) Provisional application No. 63/022,534, filed on May 10, 2020.
(60) Provisional application No. 63/022,533, filed on May 10, 2020.
(60) Provisional application No. 63/013,450, filed on Apr. 21, 2020.
(60) Provisional application No. 63/010,578, filed on Apr. 15, 2020.
(60) Provisional application No. 63/010,626, filed on Apr. 15, 2020.
(60) Provisional application No. 63/002,209, filed on Mar. 30, 2020.
(60) Provisional application No. 62/991,039, filed on Mar. 17, 2020.

(51) Int. Cl. B01L 3/00

(52) U.S. Cl. CPC B01L 3/502738; B01L 2300/044; B01L 2300/049; B01L 3/502707

(57) ABSTRACT

Provided herein, in some embodiments, are rapid diagnostic tests to detect one or more target nucleic acid sequences (e.g., a nucleic acid sequence of one or more pathogens) having a seal component. In some embodiments, the pathogens are viral, bacterial, fungal, parasitic, or protozoan pathogens, such as SARS-CoV-2 or an influenza virus. Further embodiments provide methods of detecting genetic abnormalities. Diagnostic tests comprising a sample-collecting component, one or more reagents (e.g., lysis reagents, nucleic acid amplification reagents), a seal component, and a detection component (e.g., a component comprising a lateral flow assay strip and/or a colorimetric assay) are provided.

### RELATED APPLICATIONS

This application claims the benefit under 35 U.S.C. § 119(e) of U.S. Provisional Application Ser. No. 62/991,039, filed Mar. 17, 2020, U.S. Provisional Application Ser. No. 63/002,209, filed Mar. 30, 2020, U.S. Provisional Application Ser. No. 63/010,578, filed Apr. 15, 2020, U.S. Provisional Application Ser. No. 63/010,626, filed Apr. 15, 2020, U.S. Provisional Application Ser. No. 63/013,450, filed Apr. 21, 2020, U.S. Provisional Application Ser. No. 63/022,534, filed May 10, 2020, U.S. Provisional Application Ser. No. 63/022,533, filed May 10, 2020, U.S. Provisional Application Ser. No. 63/036,887, filed Jun. 9, 2020, U.S. Provisional Application Ser. No. 63/081,201, filed Sep. 21, 2020, U.S. Provisional Application Ser. No. 63/065,131, filed Aug. 13, 2020, U.S. Provisional Application Ser. No. 63/059,928, filed Jul. 31, 2020, U.S. Provisional Application Ser. No. 63/068,303, filed Aug. 20, 2020, U.S. Provisional Application Ser. No. 63/027,859, filed May 20, 2020, U.S. Provisional Application Ser. No. 63/027,874, filed May 20, 2020, U.S. Provisional Application Ser. No. 63/027,890, filed May 20, 2020, U.S. Provisional Application Ser. No. 63/027,864, filed May 20, 2020, U.S. Provisional Application Ser. No. 63/027,878, filed May 20, 2020, U.S. Provisional Application Ser. No. 63/027,886, filed May 20, 2020, U.S. Provisional Application Ser. No. 63/034,901, filed Jun. 4, 2020, U.S. Provisional Application Ser. No. 63/053,534, filed Jul. 17, 2020, and U.S. Provisional Application Ser. No. 63/061,072, filed Aug. 4, 2020, each of which is hereby incorporated by reference in its entirety.

### FIELD

The present invention generally relates to diagnostic devices, systems, and methods for detecting the presence of a target nucleic acid sequence.

### BACKGROUND

The ability to rapidly diagnose diseases—particularly highly infectious diseases—is critical to preserving human health. As one example, the high level of contagiousness, the high mortality rate, and the lack of a treatment or vaccine for the coronavirus disease 2019 (COVID-19) have resulted in a pandemic that has already infected millions and killed hundreds of thousands of people. The existence of rapid, accurate COVID-19 diagnostic tests could allow infected individuals to be quickly identified and isolated, which could assist with containment of the disease. In the absence of such diagnostic tests, COVID-19 may continue to spread unchecked throughout communities.

### SUMMARY

Provided herein are a number of diagnostic tests useful for detecting target nucleic acid sequences. The tests, as described herein, are able to be performed in a point-of-care (POC) setting or home setting without specialized equipment. In some aspects, the disclosure provides a diagnostic test including a reservoir containing a solution as well as a readout element (e.g., lateral flow assay strip, colorimetric assay, etc.). The diagnostic test includes at least one seal positioned in a fluidic channel between the reservoir and the readout element, where the seal prevents fluid flow from the reservoir to the readout element until the seal is opened.

In some embodiments, a diagnostic test includes a first reservoir for containing a first solution, a readout element, and a seal positioned between the first reservoir and the readout element, where puncturing the seal allows the first solution to flow from the first reservoir to the readout element. In some embodiments, the seal may be a puncturable seal that may be punctured by a puncturing tool such as a blade or needle. A puncturable seal may be formed of a metal foil, plastic film, elastomeric film, or another frangible material. In some embodiments, the seal may be a valve that is openable via an actuator.

In some embodiments, a method of performing a diagnostic test includes depositing a sample in a first reservoir containing a first solution and opening a seal on the first reservoir to allow the first solution to flow to a readout element. In some embodiments, opening the seal may include puncturing the seal.

In some embodiments, a method of manufacturing a diagnostic test includes filling a first reservoir with a first solution, where the first reservoir is disposed in a housing, placing a readout element in the housing, and placing a seal positioned between the first reservoir and the readout element, where the seal is configured to allow the first solution to flow from the first reservoir to the readout element when punctured.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIGS. 1A-1B show, according to some embodiments, a detection component including a first reservoir and one embodiment of a puncturable seal;

FIGS. 2A-2C show, according to some embodiments, a detection component including a first reservoir, a second reservoir, a first seal, and a second seal;

FIGS. 3A-3C show, according to some embodiments, a detection component including a first reservoir, a second reservoir, a third reservoir, a first seal, and a second seal;

FIGS. 4A-4B show, according to some embodiments, a detection component including a first reservoir and another embodiment of a puncturable seal;

FIGS. 5A-5B show, according to some embodiments, a detection component including a first reservoir and another embodiment of a puncturable seal;

FIGS. 6A-6B show, according to some embodiments, a detection component including a first reservoir and one embodiment of a seal configured as a septum;

FIGS. 7A-7B show, according to some embodiments, a detection component including a first reservoir and another embodiment of a seal configured as a septum;

FIGS. 8A-8B show, according to some embodiments, a detection component including a first reservoir and another embodiment of a seal configured as a valve;

FIGS. 9A-9C show, according to some embodiments, a detection component including a first reservoir and another embodiment of a seal configured as a valve including a pump;

FIGS. 10A-10C show, according to some embodiments, a detection component including a first reservoir and another embodiment of a seal configured as a valve including a pump;

FIG. 11 shows, according to some embodiments, a flow chart for a method of performing a diagnostic test;

FIG. 12 shows, according to some embodiments, a flow chart for a method of manufacturing a diagnostic test;

FIG. 13 shows, according to some embodiments, a detection component comprising a “chimney”;

FIGS. 14A-14B show diagnostic kits comprising a sample-collecting component, a reaction tube, a detection component, and a heater, according to some embodiments; and

FIG. 15 is a flow chart for one embodiment of performing a diagnostic test.

### DETAILED DESCRIPTION

Conventional nucleic acid tests for various diseases requires trained medical professional to collect samples and process those samples in a sterile environment in a laboratory. Such a process is time consuming, resulting in a delay in providing results to patients. Additionally, such tests require a patient to visit a location where a sample may be collected and transported in a sterile manner to an appropriate processing location. Travel to and from locations may risk spread of the disease being tested for and may inadvertently expose medical personnel to the disease.

In view of the above, the inventors have recognized the benefits of a rapid diagnostic test that is usable by a user who may not be a trained medical professional. In particular, the inventors have recognized the benefits of a rapid diagnostic tests employing fluid reservoirs having seals that may be easily punctured to fluidly connect various fluidic elements of the rapid diagnostic test in sequence while maintaining sterility. Such a rapid diagnostic test may allow users to perform tests at home and receive results in a rapid manner without necessarily requiring input from trained medical staff. Telemedicine, or applications may be employed to further enhance the usability of the rapid diagnostic test, such that a variety of diseases such as COVID-19, influenza, (or any target nucleic acid) may be easily and reliably tested for.

### I. Seals for Controlling Fluid Transfer in a Diagnostic Test

According to exemplary embodiments described herein, it may be desirable to selectively move solutions contained in reservoirs of a diagnostic test. In particular, the inventors have recognized that moving reagents through a diagnostic test at specific times in a sterile manner may provide accurate results in a rapid manner. The inventors have also recognized the benefits of one or more reservoirs with corresponding seals configured to ensure that at certain times, fluids are maintained separate from one another (and other parts of a device) when the seals are closed, and at other times allow fluid transmission and/or mixing when the seals are opened. A seal of exemplary embodiments described herein may enable sterile fluid flow between different fluid elements of a diagnostic test. Seals according to exemplary embodiments described herein are employed in exemplary diagnostic test components (e.g., detection components, reaction tubes, etc.), though the seals may be employed with any portion of a diagnostic test in which sealing and selective fluid communication is desirable. The seal described may be used with the exemplary test described or with other tests as appropriate.

In some embodiments, the one or more seals may be configured as puncturable seals. Puncturable seals may be formed in a breakable film composed of metal (e.g., metal foil), plastic and/or an elastomer. In some embodiments, a puncturable seal may be configured to be punctured by a puncturing tool (e.g., a blade, needle, etc.). In some embodiments the one or more seals may be configured as a valve. Valves may be configured as, but are not limited to, a septum, ball valve, flutter valve, umbrella valve, pinch valve, and check valve. In some embodiments, a valve may include an actuator that is actuatable by a user to operate the valve and/or apply pressure to a portion of a diagnostic test. Additionally, seals of a diagnostic test may take different forms and may be used in any suitable combination with one another for single or multiple fluidic elements of a diagnostic test (e.g., reservoirs). For example, a puncturable seal may be used in combination with a check valve.

In addition to the above, the inventors have recognized the benefits of one or more seals positioned between one or more reservoirs of a diagnostic test and a readout element (e.g., a lateral flow assay strip, colorimetric assay, etc.), where the reservoirs may be a part of a cartridge and/or a “chimney” detection component. Such an arrangement may allow reliable fluid transmission between the one or more reservoirs and the readout element.

In some embodiments, a diagnostic test includes a first reservoir, a readout element, and a seal positioned between the first reservoir and the readout element. The seal may be configured to allow a first solution contained in the first reservoir to flow from the first reservoir to the readout element. In some embodiments, the first reservoir, readout element, and seal may be disposed in a housing. In such an embodiment, the first reservoir may be movable relative to the seal. The first reservoir may include a needle or a blade configured to puncture the seal when the first reservoir is moved toward the seal. In some embodiments, the seal may be disposed in the first reservoir, and the first reservoir may be movable relative to a housing of the diagnostic test. According to this embodiment, the housing may include a blade or needle configured to puncture the seal when the first reservoir is moved toward the blade or needle (i.e., against the housing). According to exemplary embodiments described herein, the seal may be formed of a frangible or otherwise breakable material. For example, a puncturable seal may be a metal foil, a plastic film, or an elastomeric film that is puncturable. In some embodiments, a puncturable seal may form a cap or lid of the first reservoir. In some embodiments, the puncturable seal may be punctured by a swab or other sample containing arrangement.

According to exemplary embodiments described herein, a diagnostic test includes a plurality of reservoirs fluidly connected to a readout element (e.g., a lateral flow assay strip, colorimetric assay, etc.). The reservoirs may each include a solution which is configured to be eventually transferred to the readout element. In some embodiments, a first reservoir may include a first solution and a second reservoir may include a second solution, where each of the first solution and second solution are configured to be transferred to a third reservoir and/or readout element. Seals may be positioned in a fluidic channel between the first reservoir and the second reservoir, so that the solutions contained in the first and second reservoirs are not able to flow to the third reservoir and/or readout element until the seals are punctured or otherwise opened. Accordingly, the solutions may be released in sequence or at a specific timing by puncturing or opening the seals associated the first and second reservoirs. Of course, solutions contained in reservoirs may be released and allowed to flow to other reservoirs and/or a readout element, as the present disclosure is not so limited.

In some embodiments, a diagnostic test may include a first reservoir, a readout element, and a seal configured as a valve. The valve may be switched between a closed state where a solution in the first reservoir is not able to flow to the readout element and an open state where the solution is able to flow to the readout element. In some embodiments, movement of the first reservoir relative to the valve may open the valve. For example, in some embodiments the valve may be a septum valve that is opened by the first reservoir when the first reservoir is moved against the valve. In other embodiments, the valve may be configured as any selected from a group of a ball vale, a flutter valve, an umbrella valve, a pinch valve, or a check valve. Of course, any suitable valve may be employed in diagnostic tests according to exemplary embodiments described herein, as the present disclosure is not so limited. In some embodiments, a diagnostic test may include an actuator coupled to the valve configured to open and/or close the valve. In some embodiments, the actuator may be configured as a handle operable by a user that switches the valve between an open and closed state. In other embodiments, the actuator may be configured as a plunger or pump configured to apply positive or negative pressure (e.g., a vacuum) to the reservoir. For example, in some embodiments a plunger may be employed in combination with a cylinder to form a positive displacement pump. As another example, in some embodiments the actuator may be configured as a squeeze tube that may be pressurized by a user. Of course, any suitable actuator may be used to apply pressure to a portion of a fluidic channel of a rapid diagnostic test and/or open or close a valve, as the present disclosure is not so limited.

According to exemplary embodiments described herein, a method of using a diagnostic test may including depositing a sample in a first reservoir containing a first solution and puncturing a seal on the first reservoir to allow the first solution to flow to a readout element. In some embodiments, the method may also include puncturing a second seal of a second reservoir to allow a second solution contained in the second reservoir to flow to the later flow assay strip. Puncturing the first seal and/or second seal may include moving the first reservoir and/or second reservoir, respectively. For example, in cases where the seals are positioned in the reservoirs, a puncturing tool on a housing of the diagnostic test such as a blade or needle may puncture the seals of the reservoirs when the reservoirs are moved against the housing. In cases where the seals are positioned in the housing, a puncturing tool may be positioned on each of the reservoirs and may puncture seals positioned in the housing when the reservoirs are moved against the housing. In some embodiments, the flow of the first solution and second solution may be complemented by pumping the first and second solution to the readout element. For example, a plunger and cylinder or a squeeze tube may be employed to pressurize and drive the solutions to the readout element. Of course, in other embodiments the first and second solutions may flow passively (e.g., via gravity or capillary action) to the readout element. In some embodiments, puncturing a seal may include opening a valve by movement of a reservoir, operation of an actuator, and/or pressurization of a portion of a fluidic channel.

According to exemplary embodiments described herein, a method of manufacturing a diagnostic test includes filling a first reservoir with a first solution, where the first reservoir is disposed in a housing, and placing a readout element in the housing. The method may also include placing a seal positioned in a fluidic channel between the first reservoir and the readout element, where the seal is configured to allow the first solution to flow from the first reservoir to the readout element when punctured. In some embodiments, the method may also include filling a second reservoir with a second solution, where the second reservoir is also positioned in the housing, and placing a second seal between the second reservoir and the readout element. The first solution may be a lysis solution and the second solution may be a buffer solution. In some embodiments, the method may further include placing at least one puncturing tool such as a needle or blade in the housing such that the first seal and the second seal are punctured when the first reservoir and second reservoir are moved against the housing, respectively. In some embodiments, the at least one puncturing tool may instead be placed on the reservoirs and may be configured to puncture a seal disposed in the housing.

A. Diagnostic Test Detection Component Including Puncturable Seal

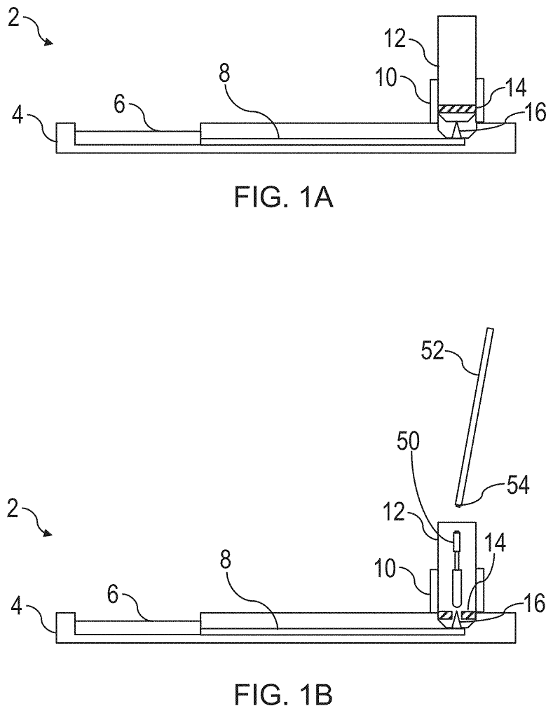

FIGS. 1A-1B show, according to some embodiments, a diagnostic test cartridge 2 including a reservoir 12 and one embodiment of a puncturable seal 14. As shown in FIG. 1A, the cartridge includes a housing 4 containing a lateral flow assay strip 6 and a fluidic channel 8. The fluidic channel fluidly connects the lateral flow assay strip 6 to the reservoir 12. According to the embodiment of FIGS. 1A-1B, the reservoir is slidably disposed in a cavity 10 formed in the housing 4. Accordingly, the reservoir is movable relative to the housing 4 in the cavity 10. As shown in FIG. 1A, the seal 14 is configured as a puncturable seal. The seal may be formed of metal foil, a plastic film, an elastomeric film, or another suitable frangible material. The housing 4 includes a puncturing tool 16 (e.g., a blade or needle) configured to selectively puncture the seal 14. In particular, as shown in the transition between the states shown in FIG. 1A and FIG. 1B, the reservoir is moved against (i.e., toward) the housing 4 into the cavity so that the puncturing tool 16 is brought into contact with the seal 14. As force is applied to the reservoir in this state, the puncturing tool may puncture the seal 14, thereby allowing a solution contained in the first container to flow through the fluidic channel 8 to the lateral flow assay strip 6. According to the embodiment of FIGS. 1A-1B, the solution may flow from the reservoir 12 to the lateral flow assay strip 6 passively (e.g., by gravity or capillary action).

As shown in FIG. 1B, the reservoir 12 is configured to receive a sample swab 50. The swab 50 may be a breakable swab, where the head of the swab is breakable from a handle 52 at a breakable portion 54. The swab 50 may be configured to collect a sample from a subject. The first reservoir may contain a buffer solution and/or a lysis solution configured to react with the sample so that one or more target nucleic acid sequences may be detected by the lateral flow assay strip. The reservoir 12 may be sized and shaped to fully receive the swab 50. Accordingly, a sample swab may be easily deposited in the reservoir 12 with the handle 52 and then detached from the handle 52 by breaking the swab off at the breakable portion 54. Once the swab 50 is deposited in the reservoir 12, the reservoir may be sealed with a cap (not shown) and allowed to incubate before the seal 14 is punctured.

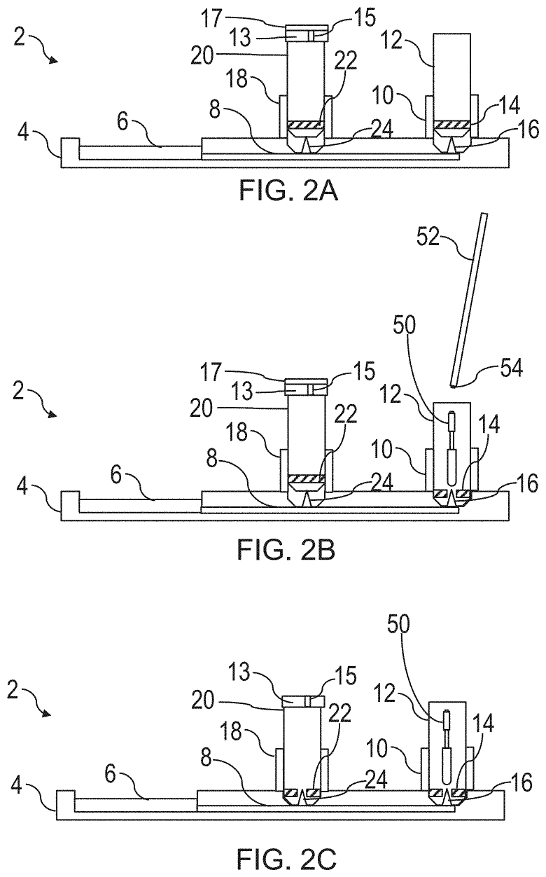

FIGS. 2A-2C show, according to some embodiments, a diagnostic test cartridge 2 including a first reservoir 12, a second reservoir 20, a first seal 14, and a second seal 22. As shown in FIGS. 2A-2C and similar to the embodiment of FIGS. 1A-1B, the cartridge includes a housing 4 holding a lateral flow assay strip 6. The lateral flow assay strip is fluidly connected to both the first reservoir and the second reservoir via fluidic channel 8. According to the embodiment of FIGS. 2A-2C, the first reservoir 12 is slidably disposed in a first cavity 10 formed in the housing 4. Likewise, the second reservoir 20 is slidably disposed in a second cavity 18 formed in the housing 4. According to the embodiment of FIGS. 2A-2C, the first seal 14 and second seal 22 are configured as puncturable seals. The seals may be formed of metal foil, a plastic film, an elastomeric film, or another suitable frangible material. The housing 4 includes a first puncturing tool 16 (e.g., a blade or needle) configured to selectively puncture the first seal 14 and a second puncturing tool 24 configured to selectively puncture the second seal 22. In particular, as shown in the transition between the states shown in FIG. 2A and FIG. 2C, the first and second reservoirs are moved against (i.e., toward) the housing 4 into their respective cavities 10, 18 so that the first puncturing tool 16 and second puncturing tool 24 are brought into contact with the respective seals to puncture the seals. Once the seals are punctured, the solutions contained in each of the reservoirs may be able to flow to the lateral flow assay strip 6. According to the embodiment of FIGS. 2A-2C, the solution may flow from the reservoirs 12, 20 to the lateral flow assay strip 6 passively (e.g., by gravity or capillary action).

In the embodiment of FIGS. 2A-2C, the second reservoir 20 includes a semi-permeable vent 15 formed in a cap 13. The semi-permeable vent may allow fluid to enter or exit the second reservoir compared to a reservoir without a semi-permeable vent. That is, the semi-permeable vent may allow air to exit the second reservoir as a sample mixture from the first reservoir 12 flows into the second reservoir 20 to replace any space vacated by the air. Likewise, the vent may allow air to enter the second reservoir as the sample mixture flows out of the second reservoir to replace any space vacated by the sample mixture. Accordingly, the semi-permeable vent may mitigate the effects of any vacuum formed in a headspace inside of the second reservoir. The semi-permeable vent may be air-permeable but not liquid permeable, such that fluid is not able to flow out of the semi-permeable vent and no liquids or other contaminants are able to flow into the second reservoir. In some embodiments as shown in FIGS. 2A-2C, the cap 13 includes a removable cover 17 configured to seal the semi-permeable vent 15. The removable cover may prevent any air from entering the second reservoir or escaping from the second reservoir. Such an arrangement may be beneficial during a heating process or a storage process of the cartridge 2. In some embodiments, the removable cover may be removed from the cap by peeling the removable cover off. Of course, any suitable cover may be employed and removed in any suitable manner, as the present disclosure is not so limited.

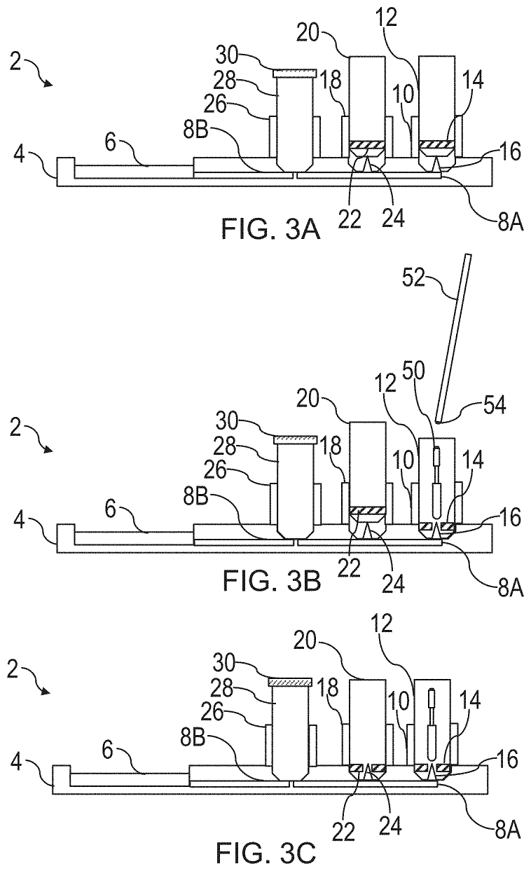

While a cap including a semi-permeable vent is shown and described with reference to the embodiment of FIGS. 2A-2C, it should be appreciated that any reservoir or tube according to exemplary embodiments herein may include a cap having a semi-permeable vent. Such semi-permeable vents may allow a user to selectively control flow of fluid through a cartridge by manipulating the ability of air to enter or exit a reservoir. Of course, any combination of caps and uncapped reservoirs may be employed, as the present disclosure is not so limited. Additionally, in some embodiments caps without semi-permeable vents may be employed to control the flow a fluid through a cartridge, as will be discussed further with reference to FIGS. 3A-3C. In some embodiments, a semi-permeable vent may be formed in a reservoir wall as opposed to a cap, as the present disclosure is not so limited. In some embodiments, a semi-permeable vent may include a check valve to allow one-way passage of air through the semi-permeable vent. For example, a semi-permeable vent may allow air to enter a reservoir to avoid formation of any vacuum inside of the reservoir but may not allow air to escape the reservoir.

According to the process shown through FIGS. 2A-2C, the first reservoir 12 may be configured to receive a swab 50 containing a sample. In some embodiments, the first reservoir may contain a lysis solution and the second reservoir 20 may contain a buffer solution. As shown in FIG. 2B, the first seal 14 may be punctured first to allow the lysis solution to enter the fluidic channel 8 before the second seal 22 is punctured (see FIG. 2C). As shown in FIG. 2C, the second reservoir 20 is moved into the second puncturing tool 24 to puncture the second seal 22. Additionally, the removable cover has been removed from the cap 13, allowing air to transfer into or out of the second reservoir 20 via the semi-permeable vent 15. Of course, depending on the particular sample and solutions used with the cartridge, the seals 14, 22 may be punctured in any suitable order or concurrently, as the present disclosure is not so limited.

FIGS. 3A-3C show, according to some embodiments, a diagnostic test cartridge including a first reservoir 12, a second reservoir 20, a third reservoir 28, a first seal 14, and a second seal 22. According to the embodiment of FIGS. 3A-3C, the cartridge is similar to the embodiment of FIGS. 2A-2C, where the cartridge includes a housing 4 holding a lateral flow assay strip 6. Additionally, the first reservoir 12 and second reservoir 20 each include respective seals, 14, 22 that are configured to be punctured by corresponding puncturing tools 16, 24. The first and second reservoirs are disposed in cavities 10, 18 of the housing so that movement of the reservoirs may be used to puncture the seals concurrently or in sequence. However, in contrast to the embodiment of FIGS. 2A-2C, the embodiment of FIGS. 3A-3C includes a first fluidic channel 8A and a second fluidic channel 8B. The first fluidic channel fluidly connects the first and second reservoirs to the third reservoir 28 which is positioned in a third cavity 26 of the housing 4. The second fluidic channel connects the third reservoir to the lateral flow assay strip. Accordingly, the first and second reservoirs are not directly connected to the lateral flow assay strip 6, and any fluid contained in the first or second reservoir flows through the third reservoir 28 to reach the lateral flow assay strip.

As shown in FIGS. 3A-3C, the third reservoir includes a cap 30 which may, in some embodiments, be removed and/or broken to control the flow of fluid from the third reservoir to the lateral flow assay strip 6. That is, in some embodiments, when the cap 30 is in place fluid may not be able to flow into the third reservoir 28 as the air and/or fluid in the third reservoir may be trapped within the third reservoir. According to such an embodiment, removing or piercing the cap 30 may allow pressure transfer between the third reservoir and atmosphere, allowing fluid to flow into and also exit the third reservoir (e.g., toward the lateral flow assay strip 6). In some embodiments, the cap 30 may comprise a hydrophobic filter configured to allow air pressure transfer between the third reservoir and atmosphere, while preventing ingress or egress of fluid through the cap. In some embodiments, the cap 30 may be configured to be peeled back by a user to allow fluid to flow into and/or exit the third reservoir. Of course, any suitable arrangement may be employed for a cap such as the cap 30 of the third reservoir 28, as the present disclosure is not so limited.

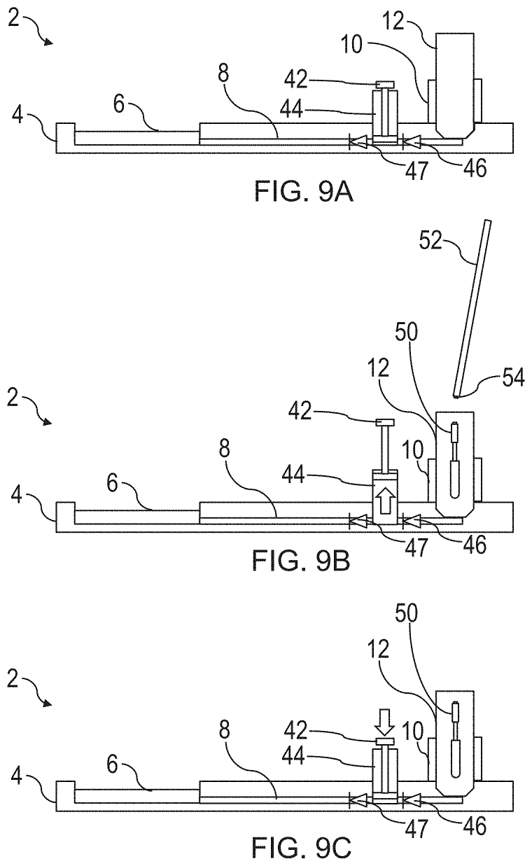

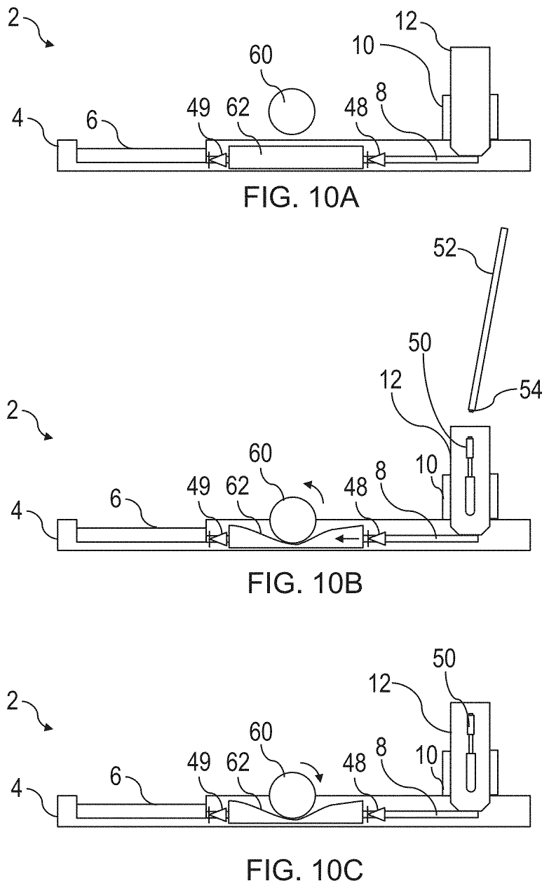

One embodiment of a process of using a diagnostic test cartridge is shown in FIGS. 3A-3C. As shown in FIG. 3B, a sample swab 50 may be deposited in the first reservoir 12. Once the sample is deposited in the first reservoir, the sample may be allowed to incubate for a predetermined period of time. In some embodiments, the sample may be heated during incubation. Once the sample has incubated, the first reservoir may be moved toward the housing 4 so that a first puncturing tool 16 punctures the first seal 14. Accordingly, the first solution from the first reservoir may flow to the third reservoir 28 through the first fluidic channel 8A. Once the first seal is punctured as shown in FIG. 3B, the second reservoir 20 may be moved against the housing to puncture the second seal 22 with a second puncturing tool 24. Once the second seal is punctured, a second solution inside of the second reservoir may flow to the third reservoir 28 through the first fluidic channel. In some embodiments, the cap 30 of the third reservoir may then be removed or punctured to allow the combined first solution and second solution to flow to the lateral flow assay strip 6 through the second fluid channel 8B. In some embodiments, rather than manipulating or puncturing the cap 30, a pump may be used to control the flow of fluid from the third reservoir to the lateral flow assay strip. Likewise, in some embodiments one or more pumps may be employed to control the flow of the first solution and the second solution to the third reservoir. Exemplary embodiments of pumps that may be employed to control the flow of fluids through the cartridge are described further with reference to FIGS. 9A-10C.

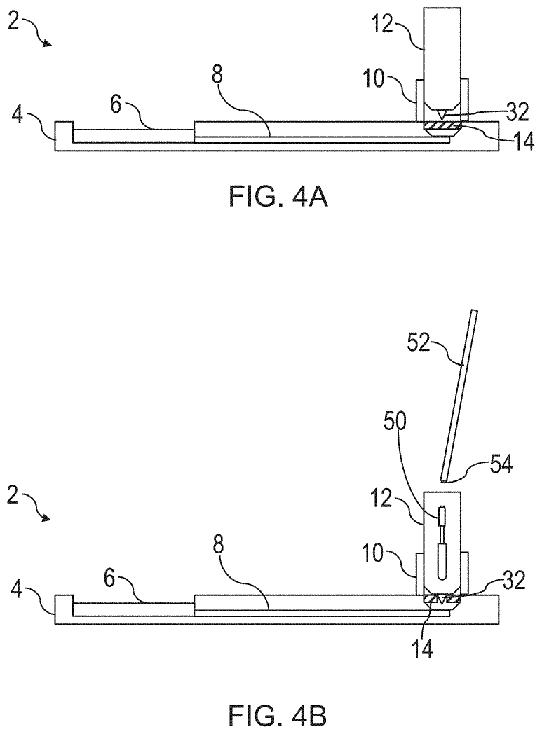

FIGS. 4A-4B show, according to some embodiments, a diagnostic test cartridge 2 including a reservoir 12 and another embodiment of a puncturable seal 14. The cartridge 2 includes a housing 4 holding a lateral flow assay strip 6 and a fluidic channel 8. The fluidic channel 8 connects the lateral flow assay strip 6 to the reservoir. Like previously described embodiments, the reservoir 12 is slidably disposed in a cavity 10 formed in the housing 4. However, in contrast to prior embodiments, the reservoir 12 includes a puncturing tool 32 (i.e., a blade or needle) and the seal 14 is positioned in the housing 4. Accordingly, rather than the housing 4 puncturing a seal positioned in the reservoir 12, the reservoir 12 punctures a seal 14 positioned in the housing 4. As shown in FIG. 4B when compared to FIG. 4A, movement of the reservoir against the housing 4 punctures the seal 14 to bring the solution contained in the reservoir into fluid communication with the fluidic channel 8 and the lateral flow assay strip 6.

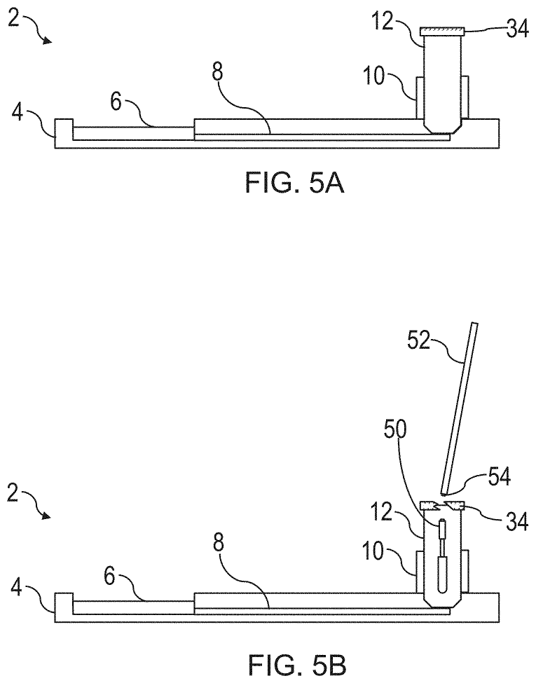

FIGS. 5A-5B show, according to some embodiments, a diagnostic test cartridge 2 including a reservoir 12 and another embodiment of a puncturable seal 34. As shown in FIGS. 5A-5B, the cartridge 2 includes a housing 4 holding a lateral flow assay strip 6 and a fluidic channel 8. The fluidic channel 8 connects the lateral flow assay strip 6 to the reservoir 12. Unlike prior embodiments, the reservoir 12 is disposed in a cavity 10 formed in the housing 4 but is not slidable relative to the cavity. Instead, the seal 34 of the reservoir 12 is configured as a puncturable cap. The puncturable cap may maintain a predetermined pressure inside of the reservoir 12, so that a solution inside of the reservoir does not flow into the fluidic channel 8. That is, if air is not able to flow into the reservoir to replace a volume of solution flowing out of the reservoir into the fluid channel, the solution is not able to flow into the fluidic channel 8 Accordingly, once the seal 34 is punctured, the seal may no longer maintain the pressure inside of the reservoir and the solution may flow into the fluidic channel and on to the lateral flow assay strip. In some embodiments as shown in FIG. 5B, the swab 50 may be used to puncture the seal 34 when the swab 50 is pressed against the seal 34 with the handle 52. Of course, the seal 34 may be punctured with another suitable tool, as the present disclosure is not so limited.

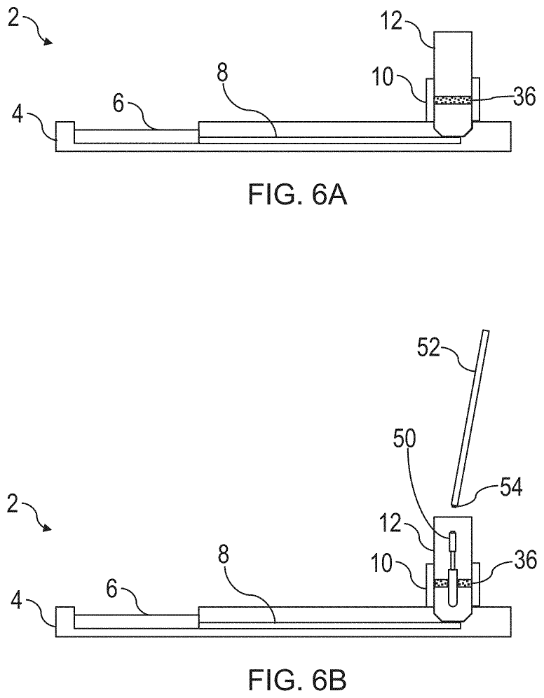

FIGS. 6A-6B show, according to some embodiments, a diagnostic test cartridge 2 including a reservoir 12 and one embodiment of a seal configured as a septum 36. As shown in FIGS. 6A-6B, the cartridge 2 includes a housing 4 holding a lateral flow assay strip 6 and a fluidic channel 8. The fluidic channel 8 fluidly connects the lateral flow assay strip 6 to the reservoir 12. As shown in FIGS. 6A-6B, the reservoir 12 includes a septum 36. Rather than being destructively opened, the septum is openable when a threshold force is applied to the septum. As shown in FIG. 6B, the swab 50 may be used to open and hold open the septum 36. Of course, the septum may be opened with any suitable tool, as the present disclosure is not so limited. In some embodiments, pressure may be applied to the reservoir (e.g., a positive pressure) to open the septum. That is, once a threshold pressure is reached, the septum valve may be opened by the pressurized solution itself. Accordingly, a pump or actuator may be used to selectively open the septum 36.

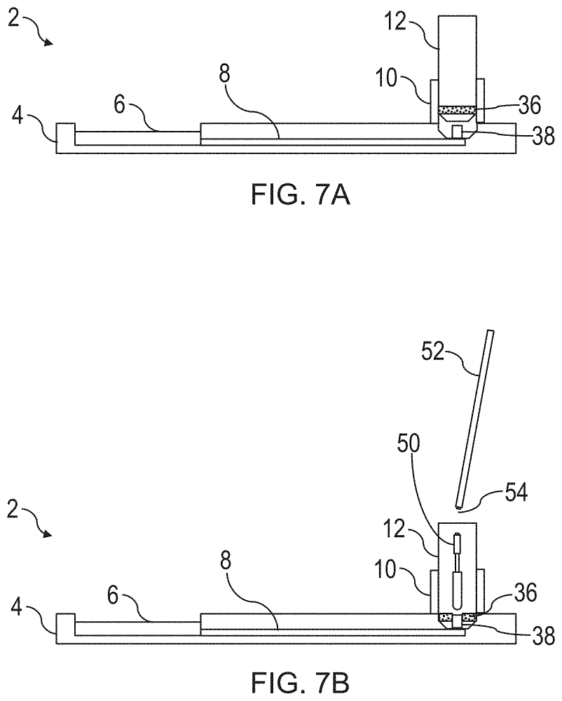

FIGS. 7A-7B show, according to some embodiments, a diagnostic test cartridge 2 including a reservoir and another embodiment of a seal configured as a septum 36. Like the embodiment of FIGS. 6A-6B, the embodiment of FIGS. 7A-7B includes a housing 4 holding a lateral flow assay strip 6 and a fluidic channel 8. The fluidic channel 8 fluidly connects the lateral flow assay strip 6 to the reservoir 12. Like the prior embodiment, the reservoir 12 includes a septum 36 openable when a threshold force is applied to the septum. In contrast to the prior embodiment, the reservoir 12 is slidably disposed in a cavity 10 formed in the housing 4. A protrusion 38 formed in the housing is configured to engage and open the septum 36 when the reservoir 12 is moved against the protrusion.

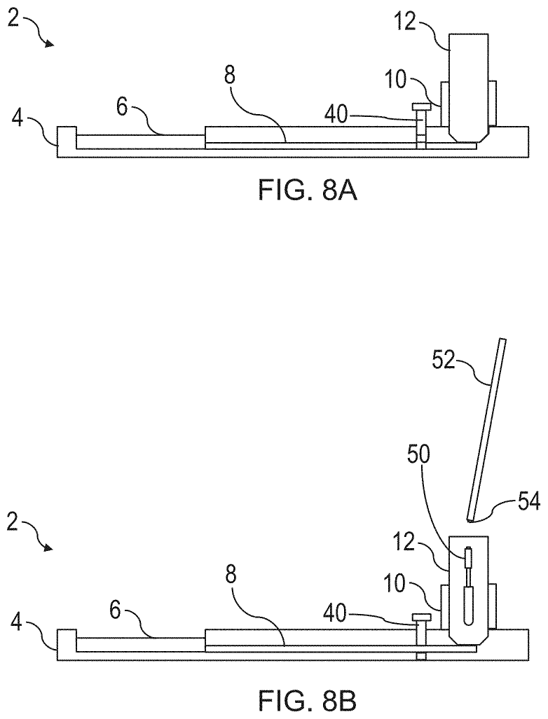

FIGS. 8A-8B show, according to some embodiments, a diagnostic test cartridge 2 including a reservoir 12 and another embodiment of a seal configured as a valve 40. Like the embodiments of FIGS. 6A-7B, the embodiment of FIGS. 8A-8B includes a housing 4 holding a lateral flow assay strip 6 and a fluidic channel 8. The fluidic channel 8 fluidly connects the lateral flow assay strip 6 to the reservoir 12. The valve 40 is positioned along the fluidic channel 8 and is movable between an open state and a closed state. In the closed state shown in FIG. 8A, the valve 40 blocks fluid from flowing from the reservoir 12 to the lateral flow assay strip 6. In the open state shown in FIG. 8B, the valve allows fluid to flow from the reservoir 12 to the lateral flow assay strip 6. The valve may be movable by a user with an actuator such as a handle or other user interface.

FIGS. 9A-9C show, according to some embodiments, a diagnostic test cartridge 2 including a reservoir 12 and another embodiment of a seal configured as a valve including a pump. Like the embodiments of FIGS. 6A-8B, the embodiment of FIGS. 9A-9C includes a housing 4 holding a lateral flow assay strip 6 and a fluidic channel 8. The fluidic channel 8 fluidly connects the lateral flow assay strip 6 to the reservoir 12. The cartridge also includes a plunger 42 and a cylinder 44 fluidly connected to the fluidic channel 8. A first check valve 46 is positioned on one side of the cylinder, while a second check valve 47 is positioned on the opposite side of the cylinder. According to the embodiment of FIGS. 9A-9C, the first and second check valves are configured to allow fluid to flow in a single direction from the reservoir 12 to the lateral flow assay strip 6. Accordingly, as shown in FIG. 9B, when the plunger is retracted (i.e., moved away from the fluidic channel 8) solution may be drawn through the first check valve 46 from the reservoir 12 and into the cylinder 44. Next, as shown in FIG. 9C, the plunger may be depressed to pressurize the fluid. As the first check valve does not allow the fluid to flow back toward the reservoir 12, the fluid is forced through the second check valve 47 toward the lateral flow assay strip 6. In this manner, the seal is a positive displacement valve that is actuatable to move fluid toward the lateral flow assay strip 6.

FIGS. 10A-10C show, according to some embodiments, a diagnostic test cartridge 2 including a reservoir 12 and another embodiment of a seal configured as a valve including a pump. Like the embodiments of FIGS. 6A-9C, the embodiment of FIGS. 10A-10C includes a housing 4 holding a lateral flow assay strip 6 and a fluidic channel 8. The fluidic channel 8 fluidly connects the lateral flow assay strip 6 to the reservoir 12. According to the embodiment of FIGS. 10A-10C, the cartridge also includes a first check valve 48 and a second check valve 49 disposed on opposite sides of a flexible container 62 (i.e., a squeeze tube). The flexible container is configured to pressurize fluid contained therein from force received from a tool such as a roller 60. That is, the roller 60 may be used to depress the flexible container 62, thereby pressurizing any fluid inside of the container Like the embodiment of FIGS. 9A-9C, the first check valve 49 and second check valve 49 are configured to allow fluid to flow in a single direction toward the lateral flow assay strip 6. Accordingly, when the flexible container is pressurized, the fluid may be driven toward the lateral flow assay strip away from the reservoir.

In some embodiments as shown in the embodiment of FIG. 10A-10C, a flexible container may be resilient. That is, when deformed the flexible container may be elastic and biased to return to an original state. Accordingly, when the flexible container 62 is deformed by the roller as shown in FIG. 10B and rolled along the flexible container, deformed portions of the flexible container may return toward an undeformed position. As a result, this elasticity may generate a vacuum or otherwise reduced pressure in the flexible container, thereby drawing fluid from the reservoir past the first check valve 48 into the flexible container. When force is applied again to a portion of the flexible container 62 containing fluid, that fluid may be driven out of the second check valve 49 toward the lateral flow assay strip. Accordingly, the roller 60 or another suitable tube may be used to pump fluid from the first reservoir toward the lateral flow assay strip 6.

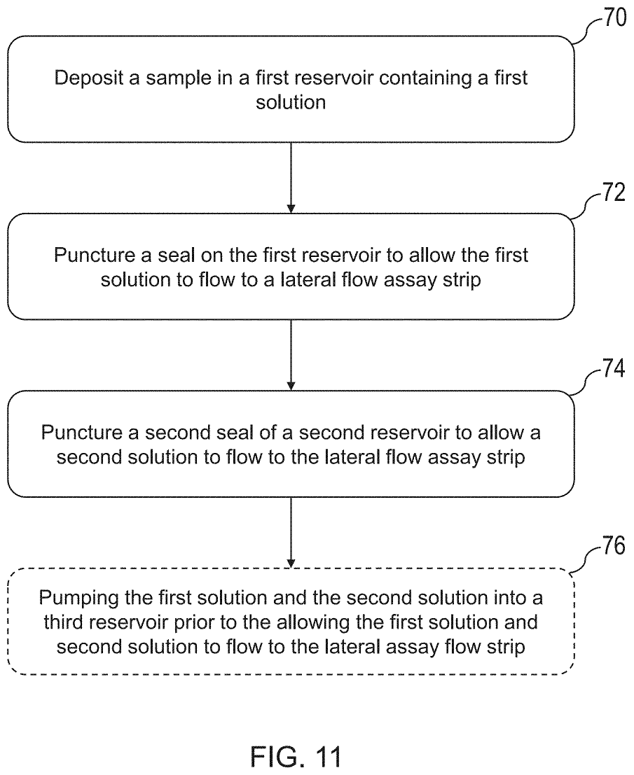

FIG. 11 shows, according to some embodiments, a flow chart for a method of performing a diagnostic test. In step 70, a sample is deposited in a first reservoir containing a first solution. The sample may be deposited as part of a swab or another suitable sample taking tool. The first solution may be a lysis solution, sample buffer solution, or other reagent for reacting with the sample. In step 72, a seal on the first reservoir is punctured to allow the first solution to flow to a lateral flow assay strip. In step 74, a second seal associated with a second reservoir is punctured to allow a second solution (e.g., a buffer solution) to flow to the lateral flow assay strip. In optional step 76, the first solution and second solution may be pumped or otherwise allowed to flow to a third reservoir prior to reaching the lateral flow assay strip. The third reservoir may be an amplification reservoir.

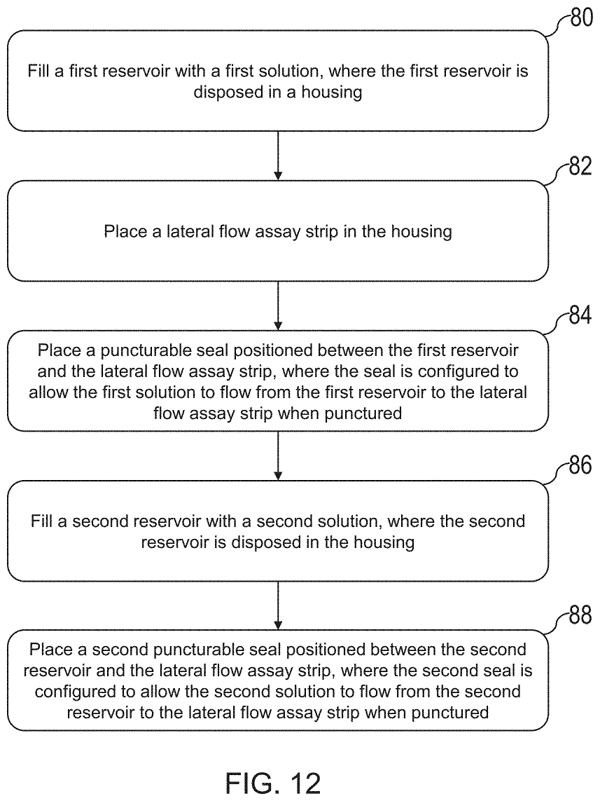

FIG. 12 shows, according to some embodiments, a flow chart for a method of manufacturing a diagnostic test. In step 80, a first reservoir is filled with a first solution (e.g., a lysis solution, sample buffer solution, etc.), where the first reservoir is disposed in a housing. The housing may include one or more fluidic channels and may accommodate a lateral assay flow strip. In step 82, a lateral flow assay strip is placed in the housing, and may be fluidly connected to one or more of the fluidic channels. In step 84, a puncturable seal may be positioned in a fluid pathway (e.g., a fluidic channel or the first reservoir) between the first reservoir and the lateral flow assay strip. In step 86, a second reservoir may be filled with a second solution (e.g., a buffer solution), where the second reservoir is also disposed in the housing. Similar to the first reservoir, in step 88 a second puncturable seal may be placed in a fluid pathway (e.g., a fluidic channel or the second reservoir) between the second reservoir and the lateral flow assay strip.

B. “Chimney” Detection Component with Puncturable Seals

In some embodiments, a diagnostic device comprises a detection component comprising a “chimney.” In certain embodiments, the “chimney” detection component comprises a chimney configured to receive a reaction tube. In certain embodiments, the “chimney” detection component comprises a puncturing component configured to puncture the reaction tube or otherwise fluidly connect the reaction tube as discussed with reference to other embodiments described herein. The puncturing component may comprise one or more blades, needles, or other elements capable of puncturing a reaction tube. In certain embodiments, the “chimney” detection component comprises a lateral flow assay strip. As described herein, the lateral flow assay strip may comprise one or more test lines configured to detect one or more target nucleic acid sequences. In some embodiments, the lateral flow assay strip further comprises one or more control lines.

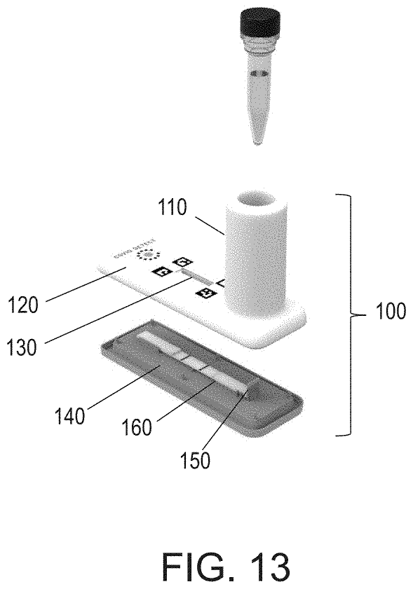

One embodiment of a “chimney” detection component is shown in FIG. 13. In FIG. 13, detection component 100 comprises chimney 110, front panel 120 comprising opening 130, and back panel 140 comprising puncturing component 150 and lateral flow assay strip 160. In some embodiments, chimney 110 and front panel 120 are integrally formed. In some embodiments, chimney 110 and front panel 120 are separately formed components that are attached to each other (e.g., via one or more screws or other fasteners, one or more adhesives, and/or one or more interlocking components). In some embodiments, front panel 120 and back panel 140 are attached to each other (e.g., via one or more screws or other fasteners, one or more adhesives, and/or one or more interlocking components). In some embodiments, front panel 120 comprises one or more markings (e.g., ArUco markers) to facilitate alignment of an electronic device (e.g., a smartphone, a tablet) with opening 130.

In operation, a reaction tube comprising fluidic contents may be inserted into chimney 110. In some embodiments, the reaction tube comprises a cap (e.g., a screw-top cap, a hinged cap) and a bottom end (e.g., a tapered or rounded bottom end). In certain cases, as shown in FIG. 13, the bottom end of the reaction tube is inserted into chimney 110 prior to the cap of the reaction tube. In certain cases, the reaction tube is inverted, and the cap of the reaction tube is inserted into chimney 110 prior to the bottom end of the reaction tube. In some embodiments, upon insertion into chimney 110, the reaction tube may lock or snap into place (or may otherwise have a secure fit) such that the reaction tube may not be easily removed from chimney 110 by the user. In certain cases, locking or snapping the reaction tube into place (or otherwise preventing easy removal of the reaction tube from chimney 110) may reduce or prevent contamination.

In some embodiments, the reaction tube may be punctured by puncturing component 150. As a result, at least a portion of the fluidic contents of the reaction tube may be deposited on a first sub-region (e.g., a sample pad) of lateral flow assay strip 160. In some cases, at least a portion of the fluidic contents of the reaction tube may be transported through lateral flow assay strip 160 (e.g., via capillary action). In some cases, for example, at least a portion of the fluidic contents of the reaction tube may flow through a second sub-region (e.g., a particle conjugate pad) of lateral flow assay strip 160 comprising a plurality of labeled particles. In some instances, the fluidic contents of the reaction tube may comprise one or more amplified nucleic acids (e.g., amplicons), and flow of at least a portion of the fluidic contents through the second sub-region (e.g., particle conjugate pad) of lateral flow assay strip 160 may result in one or more labeled amplicons. In some cases, at least a portion of the fluidic contents of the reaction tube (which may, in some instances, comprise one or more labeled amplicons) may flow through a third sub-region (e.g., a test pad) comprising one or more test lines comprising one or more capture reagents (e.g., immobilized antibodies) configured to detect one or more target nucleic acid sequences. In some instances, the formation (or lack of formation) of one or more opaque lines may indicate the presence or absence of one or more target nucleic acid sequences. In certain cases, the one or more opaque lines (if present) may be visible through opening 130 of front panel 120.

In some embodiments, a diagnostic system comprises a sample-collecting component (e.g., a swab), a reaction tube comprising one or more reagents, and a “chimney” detection component. In some embodiments, the diagnostic system further comprises a heater, as described herein.

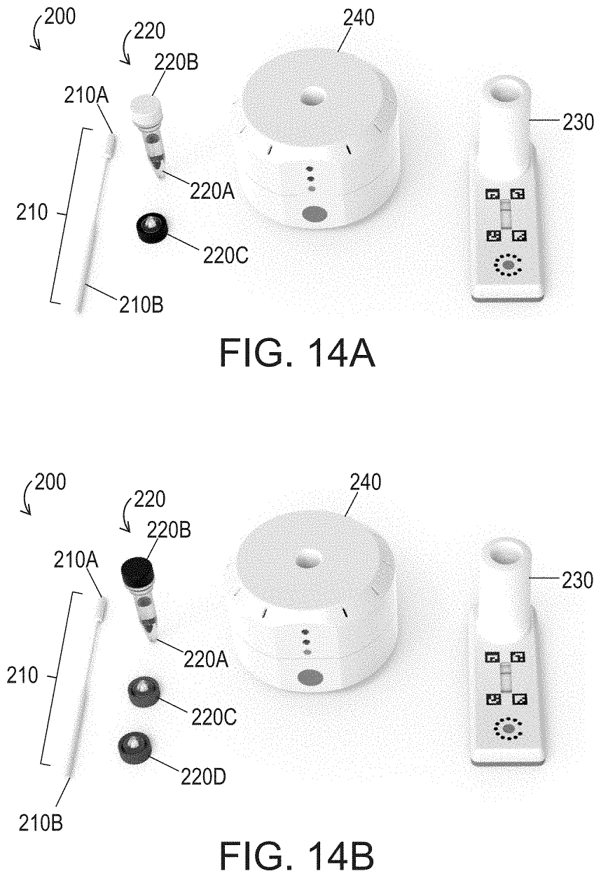

One embodiment of a diagnostic system comprising a “chimney” detection component is shown in FIG. 14A. In FIG. 14A, diagnostic system 200 comprises sample-collecting component 210, reaction tube 220, “chimney” detection component 230, and heater 240. As shown in FIG. 14A, sample-collecting component 210 may be a swab comprising swab element 210A and stem element 210B. In certain embodiment, reaction tube 220 comprises tube 220A, first cap 220B, and second cap 220C. As shown in FIG. 14A, first cap 220B and/or second cap 220C may be screw-top caps or any other types of removable caps. In certain embodiments, first cap 220B and/or second cap 220C may be airtight caps (e.g., they may fit on reaction tube 220A without any gaps and seal reaction tube 220A). In certain embodiments, second cap 220C may comprise one or more reagents (e.g., lysis reagents, nucleic acid amplification reagents, CRISPR/Cas detection reagents). In some instances, for example, second cap 220C comprises one or more blister packs comprising one or more reagents. In some embodiments, reaction tube 220 comprises fluidic contents. In certain cases, the fluidic contents of reaction tube 220 comprise a reaction buffer. In certain embodiments, the reaction buffer comprises one or more buffers (e.g., phosphate-buffered saline (PBS), Tris). In certain embodiments, the reaction buffer comprises one or more salts. Reaction tube 220 may contain any suitable volume of the reaction buffer.

In operation, a user may collect a sample using sample-collecting component 210. In some instances, for example, the user may insert swab element 210A into a nasal or oral cavity of a subject (e.g., the user, a friend or family member of the user, or any other human or animal subject). Cap 220B may be removed from tube 220A (e.g., either before or after collection of the sample), thereby exposing the fluidic contents of tube 220A, and, after collecting the sample, swab element 210A may be inserted into the fluidic contents of tube 220A. In some cases, the user may stir swab element 210A in the fluidic contents of tube 220A for a period of time (e.g., at least 10 seconds, at least 20 seconds, at least 30 seconds). In certain instances, swab element 210A is removed from tube 220A. In certain other instances, stem element 210B is broken and removed such that swab element 210A remains in tube 220A.

After swab element 210A and/or stem element 210B is removed from tube 220A, a cap may be placed on tube 220A. In some instances, for example, second cap 220C may be placed on tube 220A. In some cases, tube 220A and/or second cap 220C comprise one or more reagents (e.g., lysis reagents, nucleic acid amplification reagents, CRISPR/Cas detection reagents). In certain embodiments, second cap 220C comprises one or more reagents. In some instances, the one or more reagents are in solid form (e.g., lyophilized, dried, crystallized, air jetted). In some cases, for example, the one or more reagents are in the form of one or more tablets and/or pellets. In certain instances, the one or more tablets and/or pellets comprise one or more coatings (e.g., a coating of a time release material). In some instances, the one or more reagents are in liquid form.

The one or more reagents may be released into reaction tube 220A by any suitable mechanism. In some cases, the one or more reagents may be released into tube 220A by inverting (and, in some cases, repeatedly inverting) reaction tube 220. In some cases, second cap 220C comprises a seal (e.g. a foil seal) separating the one or more reagents from the contents of tube 220A, and the seal may be punctured by screwing second cap 220C onto tube 220A, by puncturing the seal with a puncturing tool, or otherwise puncturing the seal. In some cases, the user presses on a button or other portion of second cap 220C and/or twists at least a portion of second cap 220C to release the one or more reagents into tube 220A.

In some embodiments, reaction tube 220 may be inserted into heater 240. Reaction tube 220 may be heated at one or more temperatures (e.g., at least 37° C., at least 65° C.) for one or more periods of time. In some cases, heating reaction tube 220 according to a first heating protocol (e.g., a first set of temperature(s) and time period(s)) may facilitate lysis of cells within the collected sample. In a particular, non-limiting embodiment, a first heating protocol comprises heating reaction tube 220 at 37° C. for 5-10 minutes (e.g., about 3 minutes) and at 65° C. for 5-10 minutes (e.g., about 10 minutes). In some cases, heating reaction tube 220 according to a second heating protocol (e.g., a second set of temperature(s) and time period(s)) may facilitate amplification of one or more target nucleic acids (if present within the sample). In a particular, non-limiting embodiment, a second heating protocol comprises heating reaction tube 220 at 37° C. for 10-15 minutes. In some cases, the heater may comprise an indicator (e.g., a visual indicator) that a heating protocol is occurring. The indicator may indicate to a user when the reaction tube should be removed from the device.

Following heating, reaction tube 220 may be inserted into “chimney” detection component 230. Upon insertion, reaction tube 220 may be punctured by a puncturing component (e.g., a blade, a needle) of “chimney” detection component 230. In some cases, at least a portion of the fluidic contents of reaction tube 220 are deposited onto a portion of a lateral flow assay strip of “chimney” detection component 230. The fluidic contents of reaction tube 220 may flow through the lateral flow assay strip (e.g., via capillary action), and the presence or absence of one or more target nucleic acid sequences may be indicated on a portion of the lateral flow assay strip (e.g., by the formation of one or more lines on the lateral flow assay strip). In some instances, for example, the portion of the lateral flow assay strip may be visible to a user (e.g., through an opening, a clear window, etc.). In some cases, software (e.g., a mobile application) may be used to read, analyze, and/or report the results (e.g., the one or more lines of the lateral flow assay strip). In some embodiments, “chimney” detection component 230 comprises one or more markings (e.g., ArUco markers) to facilitate to facilitate alignment of an electronic device (e.g., a smartphone, a tablet) with “chimney” detection component 230.

Although FIG. 14A shows an embodiment comprising a reaction tube comprising first cap 220B and second cap 220C, other embodiments may comprise a reaction tube comprising a single cap. In some such embodiments, the single cap may be a removable cap (e.g., a screw-top cap), a permanently-attached cap (e.g., a hinged cap), or any other type of cap. In some cases, the single cap may comprise one or more reagents (e.g., lysis reagents, nucleic acid amplification reagents, CRISPR/Cas detection reagents). In some cases, the one or more reagents may be in solid form (e.g., lyophilized, dried, crystallized, air jetted). In some cases, the one or more reagents may be in liquid form. In certain embodiments, a user may perform one or more actions (e.g., inverting tube 220A, screwing the cap onto tube 220A, pressing on a button or other portion of the cap, twisting at least a portion of the cap) to release the one or more reagents into tube 220A. In some cases, the single cap may not comprise any reagents, and any necessary reagents may be present in tube 220A.

In some embodiments, a diagnostic system comprises a reaction tube comprising at least two caps that each comprise one or more reagents (e.g., lysis reagents, nucleic acid amplification reagents, CRISPR/Cas detection reagents). In certain embodiments, the one or more reagents are in solid form (e.g., lyophilized, dried, crystallized, air jetted). In some cases, the at least two caps may be used to sequentially add reagents to a reaction tube.

FIG. 14B shows an embodiment of diagnostic system 200 comprising reaction tube 220 comprising tube 220A, first cap 220B, second cap 220C, and third cap 220D. In certain cases, second cap 220C and third cap 220D each comprise one or more reagents. In some cases, second cap 220C may contain a first set of reagents (e.g., lysis reagents), and third cap 220D may comprise a second set of reagents (e.g., nucleic acid amplification reagents). In some cases, caps may have different colors to indicate that they contain different reagents. For example, in FIG. 14B, second cap 220C is red, while third cap 220D is blue. In some cases, the first set of reagents and/or the second set of reagents are in solid form (e.g., lyophilized, dried, crystallized, air jetted). In certain cases, for example, the one or more reagents are in the form of one or more tablets and/or pellets. In certain instances, the one or more tablets and/or pellets comprise one or more coatings (e.g., a coating of a time release material). In some cases, coatings of different materials and/or thicknesses may delay release of one or more reagents to an appropriate time in the reaction and may facilitate the sequential adding of different reagents. In some instances, the one or more reagents are in liquid form. In addition to reaction tube 220, diagnostic system 200 may comprise sample-collecting component 210, “chimney” detection component 230, and heater 240.

In operation, a user may collect a sample using sample-collecting component 210, as described above. Cap 220B may be removed from tube 220A (e.g., either before or after collection of the sample), thereby exposing the fluidic contents of tube 220A, and, after collecting the sample, swab element 210A may be inserted into the fluidic contents of tube 220A. In some cases, the user may stir swab element 210A in the fluidic contents of tube 220A for a period of time (e.g., at least 10 seconds, at least 20 seconds, at least 30 seconds). In certain instances, swab element 210A is removed from tube 220A. In certain other instances, stem element 210B is broken and removed such that swab element 210A remains in tube 220A.

After swab element 210A and/or stem element 210B is removed from tube 220A, a cap may be placed on tube 220A. In some instances, for example, second cap 220C may be placed on tube 220A. In some cases, second cap 220C comprises one or more reagents (e.g., lysis reagents). In some instances, the one or more reagents are in solid form (e.g., lyophilized, dried, crystallized, air jetted). In some cases, for example, the one or more reagents are in the form of one or more tablets and/or pellets. In certain instances, the one or more tablets and/or pellets comprise one or more coatings (e.g., a coating of a time release material). In some instances, the one or more reagents are in liquid form.

The one or more reagents may be released from second cap 220C into tube 220A by any suitable mechanism. In some cases, the one or more reagents may be released into tube 220A by inverting (and, in some cases, repeatedly inverting) reaction tube 220. In some cases, second cap 220C comprises a seal (e.g. a foil seal) separating the one or more reagents from the contents of tube 220A, and the seal may be punctured by screwing second cap 220C onto tube 220A, by puncturing the seal with a puncturing tool, or otherwise puncturing the seal. In some cases, the user presses on a button or other portion of second cap 220C and/or twists at least a portion of second cap 220C to release the one or more reagents into tube 220A.

In some cases, after the one or more reagents contained in second cap 220C have been added into tube 220A, reaction tube 220 may be heated in heater 240 according to a first heating protocol. In certain embodiments, for example, heating reaction tube 220 according to the first heating protocol may facilitate lysis of cells within the collected sample. In a particular, non-limiting embodiment, a first heating protocol comprises heating reaction tube 220 at 37° C. for 5-10 minutes (e.g., about 3 minutes) and at 65° C. for 5-10 minutes (e.g., about 10 minutes).

After completion of the first heating protocol, second cap 220C may be removed from tube 220A, and third cap 220D may be placed on tube 220A. In some embodiments, third cap 220D comprises one or more reagents (e.g., nucleic acid amplification reagents). In some instances, the one or more reagents are in solid form (e.g., lyophilized, dried, crystallized, air jetted). In some cases, for example, the one or more reagents are in the form of one or more tablets and/or pellets. In certain instances, the one or more tablets and/or pellets comprise one or more coatings (e.g., a coating of a time release material). In some instances, the one or more reagents are in liquid form.

The one or more reagents may be released from third cap 220D into tube 220A by any suitable mechanism. In some cases, the one or more reagents may be released into tube 220A by inverting (and, in some cases, repeatedly inverting) reaction tube 220. In some cases, third cap 220D comprises a seal (e.g. a foil seal) separating the one or more reagents from the contents of tube 220A, and the seal may be punctured by screwing third cap 220D onto tube 220A, by puncturing the seal with a puncturing tool, or otherwise puncturing the seal. In some cases, the user presses on a button or other portion of third cap 220D and/or twists at least a portion of third cap 220D to release the one or more reagents into tube 220A.

In some cases, after the one or more reagents contained in third cap 220D have been added into tube 220A, reaction tube 220 may be heated in heater 240 according to a second heating protocol. In certain embodiments, for example, heating reaction tube 220 according to the second heating protocol may facilitate amplification of one or more target nucleic acid sequences (if present in the sample). In a particular, non-limiting embodiment, a second heating protocol comprises heating reaction tube 220 at 32° C. for 1-10 minutes (e.g., about 3 minutes), at 65° C. for 10-40 minutes, and at 37° C. for 10-20 minutes (e.g., about 15 minutes).

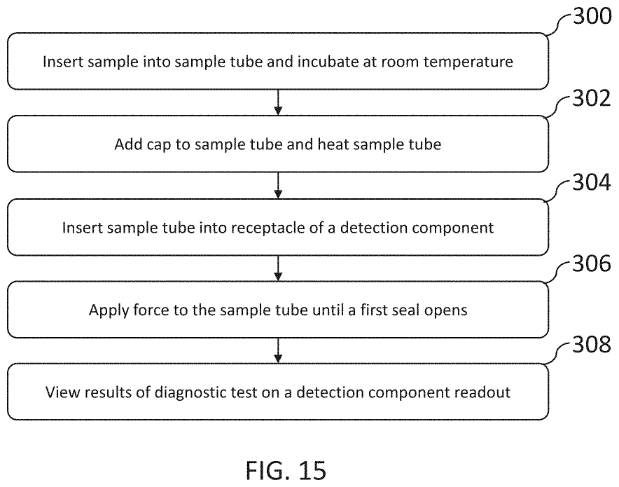

FIG. 15 is a flow chart for one embodiment of performing a diagnostic test. In step 300, a sample is inserted into a sample tube and incubated at room temperature for a first predetermined period of time. In some embodiments, the sample is taken using a swab (e.g., a nasal swab, cheek swab, etc.). In some embodiments, the sample tube may contain a buffer. In some embodiments, the first predetermined period of time is 10 minutes. In step 302, a cap is added to the sample tube and the sample tube is heated for a second predetermined period of time. In some embodiments, the cap may contain a lysis mixture which is added to the sample tube as discussed with reference to exemplary embodiments described herein. In some such embodiments, the cap may contain a blister containing the lysis mixture which may be depressed and broken by a user to deposit the lysis mixture into the sample tube. In other embodiments, adding the cap may automatically dispense the lysis mixture into the buffer and sample. In still other embodiments, a user may add the lysis mixture to the sample tube (e.g., via a pipette) prior to attaching the cap to the sample tube. In some embodiments, the sample tube may be heated at 95° C., but other temperatures are also envisioned. In some embodiments, the second predetermined period of time is approximately 3 minutes. In some embodiments, the heating is accomplished with boiling water or a fixed heat source. In some embodiments, after heating the mixture in the sample tube may be allowed to cool for 1 minute, though other time periods are also envisioned.

In step 304 of the process shown in FIG. 15, the sample tube is inserted into a receptacle (e.g., a chimney) of a detection component (e.g., a cartridge). In step 306 of the process shown in FIG. 15, force is applied to the sample tube to open a first seal. In some embodiments, the first seal may be a frangible seal configured to be punctured when the user applies force to the sample tube. In some embodiments, the first seal is a portion of a wall of the sample tube. In some embodiments, the first seal is a valve. Of course, any seal may be employed, as the present disclosure is not so limited. In some embodiments, moving the valve to the open position opening the first seal may allow the sample mixture to flow to a lateral flow assay strip disposed in the cartridge. According to some such embodiments, once the first seal is opened, the lateral flow assay strip may run automatically. In step 308, results of the test may be viewed on a readout of the detection component. In some embodiments, a user may wait 5 minutes for test results to appear on the readout. In some embodiments, the results on the lateral flow strip are interpreted using a mobile software-based application, downloadable to a smart device, such as that described herein.

According to some embodiments, a diagnostic device comprises a “chimney” detection component. In some embodiments, the “chimney” detection component comprises a chimney, a front panel, and a bottom panel comprising a lateral flow assay strip and a puncturing component. As noted above, the chimney and the front panel may be integrally formed or may be separately formed. The chimney, the front panel, and the back panel may be formed from any suitable material(s). In some cases, for example, the chimney, the front panel, and/or the back panel comprise one or more thermoplastic materials and/or metals. In some embodiments, the chimney, the front panel, and/or the back panel may be manufactured by injection molding, an additive manufacturing technique (e.g., 3D printing), and/or a subtractive manufacturing technique (e.g., laser cutting).

The chimney may have suitable dimensions to receive a reaction tube. In certain embodiments, the chimney has a height of 60 mm or less, 55 mm or less, 50 mm or less, 45 mm or less, 40 mm or less, 35 mm or less, 30 mm or less, 25 mm or less, 20 mm or less, 15 mm or less, or 10 mm or less. In some embodiments, the chimney has a height in a range from 10 mm to 20 mm, 10 mm to 30 mm, 10 mm to 40 mm, 10 mm to 50 mm, 10 mm to 60 mm, 20 mm to 30 mm, 20 mm to 40 mm, 20 mm to 50 mm, 20 mm to 60 mm, 30 mm to 40 mm, 30 mm to 50 mm, 30 mm to 60 mm, 40 mm to 50 mm, 40 mm to 60 mm, or 50 mm to 60 mm. In certain embodiments, the chimney has an inner diameter of 30 mm or less, 25 mm or less, 20 mm or less, 15 mm or less, 10 mm or less, or 5 mm or less. In some embodiments, the chimney has an inner diameter of 5 mm to 10 mm, 5 mm to 15 mm, 5 mm to 20 mm, 5 mm to 25 mm, 5 mm to 30 mm, 10 mm to 15 mm, 10 mm to 20 mm, 10 mm to 25 mm, 10 mm to 30 mm, 15 mm to 20 mm, 15 mm to 25 mm, 15 mm to 30 mm, or 20 mm to 30 mm.

In some embodiments, a chimney detection component (e.g., a cartridge) may comprises an integrated heater (e.g., a PCB heater). In some cases, detection components described herein may advantageously reduce the number of separate components in a diagnostic system. In some instances, for example, a detection component may obviate the need for a separate reaction tube and/or a separate heater.

### II. Diagnostic Test

The present disclosure provides diagnostic devices, systems, and methods for rapidly and in a point of care or home environment detecting one or more target nucleic acid sequences (e.g., a nucleic acid sequence of a pathogen, such as SARS-CoV-2 or an influenza virus) including puncturable seals. A diagnostic system, as described herein, may be self-administrable and comprise a sample-collecting component (e.g., a swab) and a diagnostic device. The diagnostic device may comprise a cartridge and/or a “chimney” detection component, according to some embodiments. In some cases, the diagnostic device comprises a assay configured to detect the presence of one or more target nucleic acids (e.g., a lateral flow assay strip, a colorimetric assay), results of which are self-readable, or automatically read by a computer algorithm. In certain embodiments, the diagnostic device further comprises one or more reagents (e.g., lysis reagents, nucleic acid amplification reagents, CRISPR/Cas detection reagents). In certain other embodiments, the diagnostic system separately includes one or more reaction tubes comprising the one or more reagents. The diagnostic device may also comprise an integrated heater, or the diagnostic system may comprise a separate heater. The isothermal amplification technique employed yields not only fast but very accurate results.

A. Diagnostic Test Applications

Diagnostic devices, systems, and methods described herein may be safely and easily operated or conducted by untrained individuals. Unlike prior art diagnostic tests, some embodiments described herein may not require knowledge of even basic laboratory techniques (e.g., pipetting). Similarly, some embodiments described herein may not require expensive laboratory equipment (e.g., thermocyclers). In some embodiments, reagents are contained within a reaction tube, a cartridge, and/or a “chimney” detection component, such that users are not exposed to any potentially harmful chemicals.

Diagnostic devices, systems, and methods described herein are also highly sensitive and accurate. In some embodiments, the diagnostic devices, systems, and methods are configured to detect one or more target nucleic acid sequences using nucleic acid amplification (e.g., an isothermal nucleic acid amplification method). Through nucleic acid amplification, the diagnostic devices, systems, and methods are able to accurately detect the presence of extremely small amounts of a target nucleic acid. In certain cases, for example, the diagnostic devices, systems, and methods can detect 1 pM or less, or 10 aM or less.

As a result, the diagnostic devices, systems, and methods described herein may be useful in a wide variety of contexts. For example, in some cases, the diagnostic devices and systems may be available over the counter for use by consumers. In such cases, untrained consumers may be able to self-administer the diagnostic test (or administer the test to friends and family members) in their own homes (or any other location of their choosing). In some cases, the diagnostic devices, systems, or methods may be operated or performed by employees or volunteers of an organization (e.g., a school, a medical office, a business). For example, a school (e.g., an elementary school, a high school, a university) may test its students, teachers, and/or administrators, a medical office (e.g., a doctor's office, a dentist's office) may test its patients, or a business may test its employees for a particular disease. In each case, the diagnostic devices, systems, or methods may be operated or performed by the test subjects (e.g., students, teachers, patients, employees) or by designated individuals (e.g., a school nurse, a teacher, a school administrator, a receptionist).

In some embodiments, diagnostic devices described herein are relatively small. In certain cases, for example, a cartridge is approximately the size of a pen or a marker. Thus, unlike diagnostic tests that require bulky equipment, diagnostic devices and systems described herein may be easily transported and/or easily stored in homes and businesses. In some embodiments, the diagnostic devices and systems are relatively inexpensive. Since no expensive laboratory equipment (e.g., a thermocycler) is required, diagnostic devices, systems, and methods described herein may be more cost effective than known diagnostic tests.

In some embodiments, any reagents contained within a diagnostic device or system described herein may be thermostabilized, and the diagnostic device or system may be shelf stable for a relatively long period of time. In certain embodiments, for example, the diagnostic device or system may be stored at room temperature (e.g., 20° C. to 25° C.) for a relatively long period of time (e.g., at least 1 month, at least 3 months, at least 6 months, at least 9 months, at least 1 year, at least 5 years, at least 10 years). In certain embodiments, the diagnostic device or system may be stored across a range of temperatures (e.g., 0° C. to 20° C., 0° C. to 37° C., 0° C. to 60° C., 0° C. to 90° C., 20° C. to 37° C., 20° C. to 60° C., 20° C. to 90° C., 37° C. to 60° C., 37° C. to 90° C., 60° C. to 90° C.) for a relatively long period of time (e.g., at least 1 month, at least 3 months, at least 6 months, at least 9 months, at least 1 year, at least 5 years, at least 10 years).

B. Target Nucleic Acid Sequences

The diagnostic devices, systems, and methods described herein may be used to detect the presence or absence of any target nucleic acid sequence (e.g., from any pathogen of interest) or multiple target nucleic acid sequences. Target nucleic acid sequences may be associated with a variety of diseases or disorders. In some embodiments, the diagnostic devices, systems, and methods are used to diagnose at least one disease or disorder caused by a pathogen. In certain instances, the diagnostic devices, systems, and methods are configured to detect a nucleic acid encoding a protein (e.g., a nucleocapsid protein) of SARS-CoV-2, which is the virus that causes COVID-19. In some embodiments, the diagnostic devices,

C. Sample Collection

In some embodiments, a diagnostic method comprises collecting a sample from a subject (e.g., a human subject, an animal subject). In some embodiments, a diagnostic system comprises a sample-collecting component configured to collect a sample from a subject (e.g., a human subject, an animal subject). Exemplary samples include bodily fluids (e.g. mucus, saliva, blood, serum, plasma, amniotic fluid, sputum, urine, cerebrospinal fluid, lymph, tear fluid, feces, or gastric fluid), cell scrapings (e.g., a scraping from the mouth or interior cheek), exhaled breath particles, tissue extracts, culture media (e.g., a liquid in which a cell, such as a pathogen cell, has been grown), environmental samples, agricultural products or other foodstuffs, and their extracts. In some embodiments, the sample comprises a nasal secretion. In certain instances, for example, the sample is an anterior nares specimen. An anterior nares specimen may be collected from a subject by inserting a swab element of a sample-collecting component into one or both nostrils of the subject for a period of time. In some embodiments, the sample comprises a cell scraping. In certain embodiments, the cell scraping is collected from the mouth or interior cheek. The cell scraping may be collected using a brush or scraping device formulated for this purpose. The sample may be self-collected by the subject or may be collected by another individual (e.g., a family member, a friend, a coworker, a health care professional) using a sample-collecting component described herein.

D. Lysis of Sample

In some embodiments, lysis is performed by chemical lysis (e.g., exposing a sample to one or more lysis reagents) and/or thermal lysis (e.g., heating a sample). Chemical lysis may be performed by one or more lysis reagents. In some embodiments, the one or more lysis reagents comprise one or more enzymes. In some embodiments, the one or more lysis reagents comprise one or more detergents. In some embodiments, cell lysis is accomplished by applying heat to a sample (thermal lysis). In certain instances, thermal lysis is performed by applying a lysis heating protocol comprising heating the sample at one or more temperatures for one or more time periods using any heater described herein. In some embodiments, a lysis heating protocol comprises heating the sample at a first temperature for a first time period.

E. Nucleic Acid Amplification

Following lysis, one or more target nucleic acids (e.g., a nucleic acid of a target pathogen) may be amplified. In some cases, a target pathogen has RNA as its genetic material. In certain instances, for example, a target pathogen is an RNA virus (e.g., a coronavirus, an influenza virus). In some such cases, the target pathogen's RNA may need to be reverse transcribed to DNA prior to amplification. In some embodiments, reverse transcription is performed by exposing lysate to one or more reverse transcription reagents. In certain instances, the one or more reverse transcription reagents comprise a reverse transcriptase, a DNA-dependent polymerase, and/or a ribonuclease (RNase). In some embodiments, DNA may be amplified according to any nucleic acid amplification method known in the art.

1) LAMP

In some embodiments, the nucleic acid amplification reagents are LAMP reagents. LAMP refers to a method of amplifying a target nucleic acid using at least four primers through the creation of a series of stem-loop structures. Due to its use of multiple primers, LAMP may be highly specific for a target nucleic acid sequence.

2) RPA

In some embodiments, the nucleic acid amplification reagents are RPA reagents. RPA generally refers to a method of amplifying a target nucleic acid using a recombinase, a single-stranded DNA binding protein, and a strand-displacing polymerase.

3) Nicking Enzyme Amplification Reaction (NEAR)

In some embodiments, amplification of one or more target nucleic acids is accomplished through the use of a nicking enzyme amplification reaction (NEAR) reaction. NEAR generally refers to a method for amplifying a target nucleic acid using a nicking endonuclease and a strand displacing DNA polymerase. In some cases, NEAR may allow for amplification of very small amplicons.

F. Molecular Switches

As described herein, a sample undergoes lysis and amplification prior to detection. In certain embodiments, one or more (and, in some cases, all) of the reagents necessary for lysis and/or amplification are present in a single pellet or tablet. In some embodiments, a pellet or tablet may comprise two or more enzymes, and it may be necessary for the enzymes to be activated in a particular order. Therefore, in some embodiments, the enzyme tablet further comprises one or more molecular switches. Molecular switches, as described herein, are molecules that, in response to certain conditions, reversibly switch between two or more stable states. In some embodiments, the condition that causes the molecular switch to change its configuration is pH, light, temperature, an electric current, microenvironment, or the presence of ions and other ligands. In one embodiment, the condition is heat. In some embodiments, the molecular switches described herein are aptamers. Aptamers generally refer to oligonucleotides or peptides that bind to specific target molecules (e.g., the enzymes described herein). The aptamers, upon exposure to heat or other conditions, may dissociate from the enzymes. With the use of molecular switches, the processes described herein (e.g., lysis, decontamination, reverse transcription, and amplification) may be performed in a single test tube with a single enzymatic tablet.

G. Detection

In some embodiments, amplified nucleic acids (i.e., amplicons) may be detected using any suitable methods. In some embodiments, one or more target nucleic acid sequences are detected using a lateral flow assay strip.

In some embodiments, the fluidic sample is introduced to a first sub-region (e.g., a sample pad) of the lateral flow assay strip. In certain embodiments, the fluidic sample subsequently flows through a second sub-region (e.g., a particle conjugate pad) comprising a plurality of labeled particles. In some cases, the particles comprise gold nanoparticles (e.g., colloidal gold nanoparticles). The particles may be labeled with any suitable label. Non-limiting examples of suitable labels include biotin, streptavidin, fluorescein isothiocyanate (FITC), fluorescein amidite (FAM), fluorescein, and digoxigenin (DIG). In some cases, as an amplicon-containing fluidic sample flows through the second sub-region (e.g., a particle conjugate pad), a labeled nanoparticle binds to a label of an amplicon, thereby forming a particle-amplicon conjugate.

In some embodiments, the fluidic sample (e.g., comprising a particle-amplicon conjugate) subsequently flows through a third sub-region (e.g., a test pad) comprising one or more test lines. In some embodiments, a first test line comprises a capture reagent (e.g., an immobilized antibody) configured to detect a first target nucleic acid. In some embodiments, a particle-amplicon conjugate may be captured by one or more capture reagents (e.g., immobilized antibodies), and an opaque marking may appear. The marking may have any suitable shape or pattern (e.g., one or more straight lines, curved lines, dots, squares, check marks, x marks).

In certain embodiments, the lateral flow assay strip comprises one or more additional test lines. In some instances, each test line of the lateral flow assay strip is configured to detect a different target nucleic acid. In some instances, two or more test lines of the lateral flow assay strip are configured to detect the same target nucleic acid. The test line(s) may have any suitable shape or pattern (e.g., one or more straight lines, curved lines, dots, squares, check marks, x marks).

In certain embodiments, the third sub-region (e.g., the test pad) of the lateral flow assay strip further comprises one or more control lines. In certain instances, a first control line is a human (or animal) nucleic acid control line. In some embodiments, for example, the human (or animal) nucleic acid control line is configured to detect a nucleic acid (e.g., RNase P) that is generally present in all humans (or animals). In some cases, the human (or animal) nucleic acid control line becoming detectable indicates that a human (or animal) sample was successfully collected, nucleic acids from the sample were amplified, and the amplicons were transported through the lateral flow assay strip. In certain instances, a first control line is a lateral flow control line. In some cases, the lateral flow control line becoming detectable indicates that a liquid was successfully transported through the lateral flow assay strip. In some embodiments, the lateral flow assay strip comprises two or more control lines. The control line(s) may have any suitable shape or pattern (e.g., one or more straight lines, curved lines, dots, squares, check marks, x marks). In some instances, for example, the lateral flow assay strip comprises a human (or animal) nucleic acid control line and a lateral flow control line.

In certain embodiments, the lateral flow assay strip comprises a fourth sub-region (e.g., a wicking area) to absorb fluid flowing through the lateral flow assay strip. Any excess fluid may flow through the fourth sub-region. As an illustrative example, a fluidic sample comprising an amplicon labeled with biotin and FITC may be introduced into a lateral flow assay strip (e.g., through a sample pad of a lateral flow assay strip). In some embodiments, as the labeled amplicon is transported through the lateral flow assay strip (e.g., through a particle conjugate pad of the lateral flow assay strip), a gold nanoparticle labeled with streptavidin may bind to the biotin label of the amplicon. In some cases, the lateral flow assay strip (e.g., a test pad of the lateral flow assay strip) may comprise a first test line comprising an anti-FITC antibody. In some embodiments, the gold nanoparticle-amplicon conjugate may be captured by the anti-FITC antibody, and an opaque band may develop as additional gold nanoparticle-amplicon conjugates are captured by the anti-FITC antibodies of the first test line. In some cases, the lateral flow assay strip (e.g., a test pad of the lateral flow assay strip) further comprises a first lateral flow control line comprising biotin. In some embodiments, excess gold nanoparticles labeled with streptavidin (i.e., gold nanoparticles that were not conjugated to an amplicon) transported through the lateral flow assay strip may bind to the biotin of the first lateral flow control line, demonstrating that liquid was successfully transported to the first lateral flow control line. In certain embodiments, for example, a fluidic sample is exposed to a reagent that undergoes a color change when bound to a target nucleic acid (e.g., viral DNA or RNA), such as with an enzyme-linked immunoassay. In some embodiments, the assay further comprises a stop reagent, such as sulfonic acid. That is, when the fluidic sample is mixed with the reagents, the solution turns a specific color (e.g., red) if the target nucleic acid is present, and the sample is positive. If the solution turns a different color (e.g., green), the target nucleic acid is not present, and the sample is negative.

H. Instructions & Software

In some embodiments, a diagnostic system comprises instructions for using a diagnostic device and/or otherwise performing a diagnostic test method. The instructions may include instructions for the use, assembly, and/or storage of the diagnostic device and any other components associated with the diagnostic system. The instructions may be provided in any form recognizable by one of ordinary skill in the art as a suitable vehicle for containing such instructions. For example, the instructions may be written or published, verbal, audible (e.g., telephonic), digital, optical, visual (e.g., videotape, DVD, etc.) or electronic communications (including Internet or web-based communications). In some embodiments, the instructions are provided as part of a software-based application. In certain cases, the application can be downloaded to a smartphone or device, and then guides a user through steps to use the diagnostic device.

In some embodiments, a software-based application may be connected (e.g., via a wired or wireless connection) to one or more components of a diagnostic system. In certain embodiments, for example, a heater may be controlled by a software-based application. In some cases, a user may select an appropriate heating protocol through the software-based application. In some cases, an appropriate heating protocol may be selected remotely (e.g., not by the immediate user). In some cases, the software-based application may store information (e.g., regarding temperatures used during the processing steps) from the heater.

In some embodiments, a diagnostic system comprises or is associated with software to read and/or analyze test results. In some embodiments, a device (e.g., a camera, a smartphone) is used to generate an image of a test result (e.g., one or more lines detectable on a lateral flow assay strip). In some embodiments, a user may use an electronic device (e.g., a smartphone, a tablet, a camera) to acquire an image of the visible portion of the lateral flow assay strip. In some embodiments, software running on the electronic device may be used to analyze the image (e.g., by comparing any lines or other markings that appear on the lateral flow assay strip with known patterns of markings). That result may be communicated directly to a user or to a medical professional. In some cases, the test result may be further communicated to a remote database server. In some embodiments, the remote database server stores test results as well as user information such as at least one of name, social security number, date of birth, address, phone number, email address, medical history, and medications.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

What is claimed is:

1. A diagnostic test comprising:
a first reservoir for containing a first solution;
a readout element; and
a seal positioned between the first reservoir and the readout element, wherein puncturing the seal allows the first solution to flow from the first reservoir to the readout element.

2. The diagnostic test of claim 1, wherein the seal is a valve.

3. The diagnostic test of claim 2, wherein the valve is a septum valve, and wherein moving the first reservoir against the seal is configured to open the septum valve.

4. The diagnostic test of claim 1, wherein the first reservoir, the readout element, and the seal are disposed in a housing, and wherein the seal is positioned on the first reservoir.

5. The diagnostic test of claim 1, wherein the first reservoir, the readout element, and the seal are disposed in a housing, and wherein the seal is positioned on the housing.

6. The diagnostic test of claim 1, further comprising:
a second reservoir containing a second solution; and
a second seal positioned between the second reservoir and the readout element, wherein puncturing the second seal allows the second solution to flow from the first reservoir to the readout element.

7. The diagnostic test of claim 6, further comprising a third reservoir fluidly connected to the readout element, wherein the third reservoir is positioned between the first reservoir and the second reservoir and the readout element.

8. A method of performing a diagnostic test, comprising:
depositing a sample in a first reservoir containing a first solution; and
opening a seal on the first reservoir to allow the first solution to flow to a readout element.

9. The method of claim 8, wherein opening the seal on the first reservoir includes puncturing the seal.

10. The method of claim 9, further comprising puncturing a second seal of a second reservoir to allow a second solution in the second reservoir to flow to the readout element.

11. The method of claim 10, wherein puncturing the seal includes applying force to the seal with a blade or a needle.

12. The method of claim 11, wherein puncturing the second seal includes applying force to the seal with a second blade or second needle.

13. A method of manufacturing a diagnostic test, comprising:
filling a first reservoir with a first solution, wherein the first reservoir is disposed in a housing;
placing a readout element in the housing; and
placing a seal positioned between the first reservoir and the readout element, wherein the seal is configured to allow the first solution to flow from the first reservoir to the readout element when punctured.

14. The method of claim 13, further comprising:
filling a second reservoir with a second solution, wherein the second reservoir is disposed in the housing; and
placing a second seal between the second reservoir and the readout element.

15. The method of claim 14, wherein the first solution is a lysis solution and the second solution is a buffer solution.

16. The method of claim 13, wherein the seal is positioned on the first reservoir.

17. The method of claim 16, further comprising placing a needle in the housing configured to puncture the seal when the first reservoir is moved against the needle.

18. The method of claim 16, further comprising placing a blade in the housing configured to puncture the seal when the first reservoir is moved against the blade.

19. The method of claim 13, wherein the seal is positioned in the housing.

20. The method of claim 19, wherein the first reservoir includes a needle or a blade configured to puncture the seal when the first reservoir is moved against the housing.
